(19) United States
(12) Patent Application Publication
(10) Pub. No.: US 2026/0165781 A1
(43) Pub. Date: Jun. 18, 2026

(54) BALLOON CATHETER WITH SPLIT ELECTRODES

(71) Applicant: Biosense Webster (Israel) Ltd., Yokneam (IL)

(72) Inventors: Assaf GOVARI, Haifa (IL); Christopher Thomas BEECKLER, Brea, CA (US); Joseph Thomas KEYES, Sierra Madre, CA (US); Kevin Justin HERRERA, West Covina, CA (US)

(73) Assignee: Biosense Webster (Israel) Ltd., Yokneam (IL)

(21) Appl. No.: 19/533,663

(22) Filed: Feb. 9, 2026

Related U.S. Application Data
(63) Continuation of application No. 17/086,164, filed on Oct. 30, 2020, now Pat. No. 12,544,128.

(51) Int. Cl. A61B 18/14; A61B 18/00; A61B 18/12

(52) U.S. Cl. CPC A61B 18/1492; A61B 18/1206; A61B 2018/0022; A61B 2018/00577; A61B 2018/00839; A61B 2018/1467; A61B 2218/002

(57) ABSTRACT

A medical apparatus includes a probe, which includes an insertion tube configured for insertion into a body cavity. A balloon is connected distally to the insertion tube and is inflated within the body with a fluid that flows into the balloon through the insertion tube. Electrodes are disposed at different respective locations on a surface of the balloon and configured to contact tissue within the body cavity, each electrode being divided into multiple segments, including at least two segments having different respective areas. An electrical signal generator applies radio-frequency (RF) signals simultaneously in parallel to the multiple segments of each electrode with an amplitude sufficient to ablate the tissue contacted by the electrode. Sensing circuitry acquires electrophysiological signals from at least one of the multiple segments of each electrode separately and independently of the other segments of the electrode.

### CROSS-REFERENCE TO RELATED APPLICATIONS

This application is a continuation of, and claims priority under 35 U.S.C. § 120 to, prior filed U.S. patent application Ser. No. 17/086,164 filed Oct. 30, 2020 (Attorney Ref. No.: BIO6373USNP1-253757.000290). The entire contents of which are hereby incorporated by reference.

### FIELD OF THE INVENTION

The present invention relates generally to medical devices, and particularly to devices and methods for ablation and sensing of physiological tissues.

### BACKGROUND

Radio-frequency ablation (RFA) is a medical procedure in which part of the electrical conduction pathways of the heart or other dysfunctional tissue are ablated using the heat generated from radio-frequency (RF) alternating current (for example in the frequency range of 350-500 kHz). The ablation is done by inserting a probe, such as a catheter, into the tissue, and applying the RF current to electrodes at the tip of the probe. The probe may also be used for acquiring electrophysiological signals for diagnostic purposes.

United States Patent Application Publication 2015/0119877 describes methods, systems, and devices for providing treatment to a tissue in body lumens. The system may include a support shaft, an expansion member coupled with a distal portion of the support shaft, and an ablation structure wrapped around the expansion member less than a circumference of the expansion member configured to engage the body lumens with varying sizes.

United States Patent Application Publication 2012/0029500 describes a catheter that includes a flexible shaft having a length sufficient to access a patient's renal artery. A treatment element at the distal end of the shaft is dimensioned for deployment within the renal artery. The treatment element includes a radially expandable structure configured to maintain positioning within the renal artery.

U.S. Pat. No. 10,653,480 describes a method of constructing an electrophysiology catheter having a flex circuit electrode assembly. The method includes providing a flex circuit having a substrate, a first conductive layer and a second conductive layer.

### SUMMARY

Embodiments of the present invention that are described hereinbelow provide improved probes for ablation and sensing, as well as methods for their production and operation.

There is therefore provided, in accordance with an embodiment of the present invention, a medical apparatus, which includes a probe. The probe includes an insertion tube configured for insertion into a body cavity of a patient, a balloon, which is connected distally to the insertion tube and is configured to be inflated within the body cavity with a fluid that flows into the balloon through the insertion tube, and a plurality of electrodes, which are disposed at different respective locations on a surface of the balloon and are configured to contact tissue within the body cavity. Each electrode is divided into multiple segments, including at least two segments having different respective areas. The medical apparatus also includes an electrical signal generator, which is configured to apply radio-frequency (RF) signals simultaneously in parallel to the multiple segments of each electrode with an amplitude sufficient to ablate the tissue contacted by the electrode. Sensing circuitry is configured to acquire electrophysiological signals from at least one of the multiple segments of each electrode separately and independently of the other segments of the electrode.

In a disclosed embodiment, the at least two segments include first and second segments having respective first and second areas, such that the first area is at least twice the second area.

In a further embodiment, the first area is at least four times the second area.

In yet a further embodiment, the balloon includes one or more irrigation apertures passing through the first segment, but not through the second segment, such that the fluid flows out of the balloon through the irrigation apertures to irrigate the tissue contacted by at least the first segment.

In a disclosed embodiment, each electrode is divided into the segments by at least one longitudinal isolation line. Additionally or alternatively, each electrode is divided into the segments by at least one latitudinal isolation line.

There is also provided, in accordance with an embodiment of the present invention, a method for medical treatment and diagnostics. The method includes providing a probe for insertion into a body cavity of a patient, wherein the probe includes an insertion tube, a balloon, which is connected distally to the insertion tube and a plurality of electrodes, which are disposed at different respective locations on a surface of the balloon, each electrode being divided into multiple segments, including at least two segments having different respective areas. The method further includes inflating the balloon within the body cavity with a fluid that flows into the balloon through the insertion tube, so that one or more of the electrodes on the surface of the inflated balloon contact tissue within the body cavity. Radio-frequency (RF) signals are applied simultaneously in parallel to the multiple segments of the one or more of the electrodes with an amplitude sufficient to ablate the tissue contacted by the electrodes. Electrophysiological signals are acquired from at least one of the multiple segments of each of the one or more of the electrodes separately and independently of the other segments of the electrodes.

The present invention will be more fully understood from the following detailed description of the embodiments thereof, taken together with the drawings in which:

### BRIEF DESCRIPTION OF THE DRAWINGS

FIG. 1 is a schematic pictorial illustration of a medical apparatus in the course of an RFA procedure, in accordance with an embodiment of the invention; and

FIG. 2 is a schematic detail view of the distal end of a combined ablation and signal acquisition catheter, in accordance with an embodiment of the invention.

### DETAILED DESCRIPTION OF EMBODIMENTS

In a radio-frequency ablation (RFA) procedure, an alternating electrical current, typically with a frequency between 350 and 500 kHz, is driven through the tissue of a subject. The electrical current is carried into the tissue through the electrodes of a catheter placed in contact with the tissue. These electrodes may also be used for diagnostic purposes, by acquiring electrophysiological signals from the tissue they are touching.

Some RFA procedures use a balloon catheter, which has a balloon at its distal end and electrodes arrayed around the surface of the balloon. The balloon is inflated within the body cavity, and the electrodes are then brought into contact with the tissue that is to be ablated. To avoid damage to the electrodes and injury to the tissue due to excessive current density, the electrodes on the balloon are typically large, for example about 5 mm².

For ablating tissue within the body, for example in the left atrium of the heart, balloons of small diameter can be used, for example with diameter less than 15 mm. Despite the small size of the balloon itself, the electrodes are large enough to be able to transfer RFA current without being damaged. In this case, the size of the electrodes precludes them from being effectively used for diagnosis, since each electrode acquires signals from a relatively large region of tissue, and at any given time this region typically generates multiple signals. The balloon could have separate electrodes for signal acquisition, but this solution may be impractical due to the small size of the balloon.

The embodiments of the present invention that are described herein address this problem by providing a probe having a balloon with segmented electrodes. An electrical signal generator applies RF signals simultaneously in parallel to multiple segments of each electrode with an amplitude sufficient to ablate the tissue contacted by the electrode. On the other hand, sensing circuitry is able to acquire electrophysiological signals from at least one of the segments of each electrode separately and independently of the other segments. Thus, the electrode has a sufficient effective area to deliver the RFA current safely, while still allowing signals to be acquired with fine spatial resolution.

In the disclosed embodiments, the probe comprises an insertion tube for insertion into a body cavity of a patient, as well as a balloon connected to the distal end of the insertion tube and inflatable with a fluid that flows into the balloon through the insertion tube. The surface of the balloon has a plurality of electrodes for contacting tissue within the body cavity, with each electrode divided into segments of unequal areas.

The electrical signal generator applies radio-frequency (RF) signals simultaneously in parallel to the segments of each electrode with an amplitude sufficient to ablate the tissue contacted by the electrode. Connecting the segments in parallel for RFA, particularly the larger segments, ensures a sufficiently large surface area in order to avoid damage to the electrode due to the RF currents.

The sensing circuitry acquires separate, independent electrophysiological signals from separate segments of each electrode. Acquiring the signals particularly from the smaller segments ensures that each segment acquires its signal from a small, localized area of the tissue.

In a further embodiment, irrigation apertures pass through the larger segments utilized for RFA, so that fluid may flow out of the balloon through the apertures to irrigate the tissue contacted by the larger segments. The smaller segments, however, may have no irrigation apertures as they are utilized mainly for signal acquisition and deliver at most a small fraction of the ablation current.

### System Description

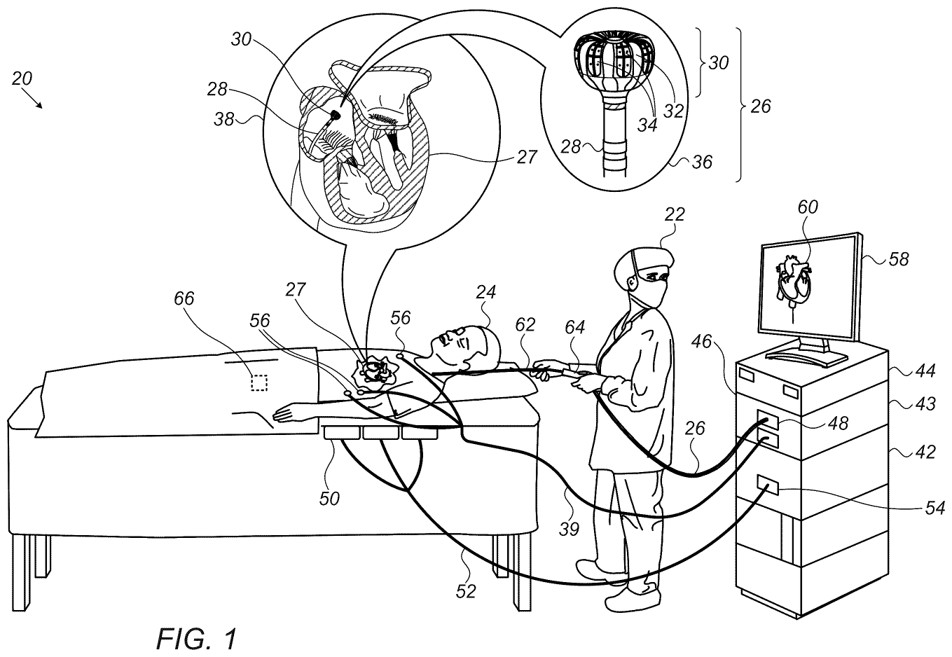

FIG. 1 is a schematic pictorial illustration of a medical apparatus 20 in the course of an RFA procedure, in accordance with an embodiment of the invention. A physician 22 performs the RFA procedure on a subject 24, using an ablation catheter 26, with further details of the catheter described hereinbelow. Physician 22 further utilizes ablation catheter 26 for acquiring electrophysiological signals from tissue of subject 24, either concurrently or alternatingly with emitting RF currents. The embodiment shown in the current figure and subsequent figures refers to an example of an RFA procedure in a chamber of a heart 27. In alternative embodiments, the RFA procedure and electrophysiological signal acquisition may be performed not only in heart 27, but also in other organs and tissue, as will be apparent to those skilled in the art after reading the present description.

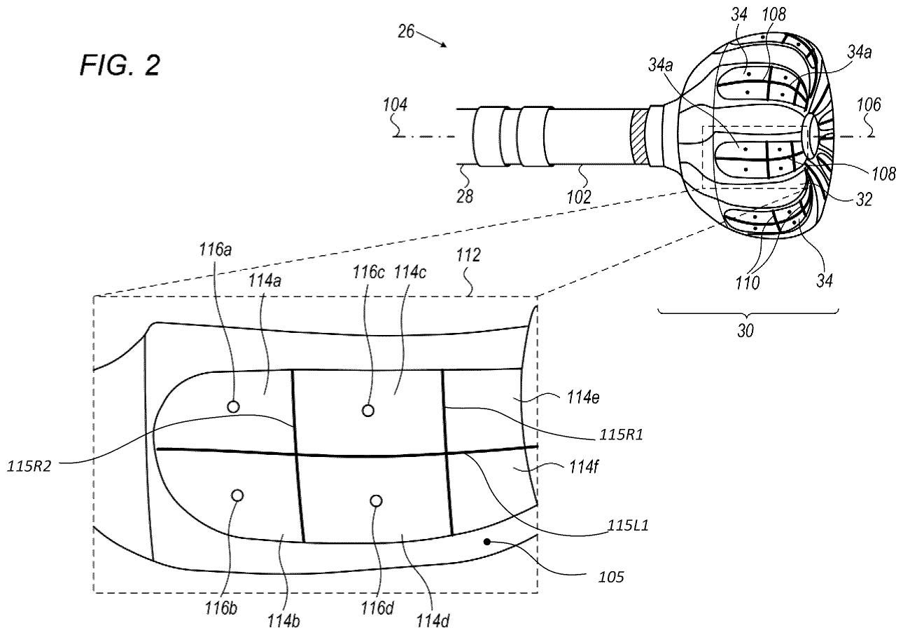

As shown in an inset 36, ablation catheter 26 comprises a shaft 28 and a distal assembly 30, wherein the shaft functions as an insertion tube for inserting the distal assembly into the chamber of heart 27. Distal assembly 30 comprises a balloon 32 with a plurality of ablation electrodes 34, wherein the electrodes have been divided into segments having unequal areas, as shown in FIG. 2. Distal assembly 30 and a part of shaft 28 are also shown in an inset 38.

Medical apparatus 20 further comprises a processor 42, sensing circuitry 43, and an electrical signal generator 44, typically residing in a console 46. The processor, the sensing circuitry, and the signal generator may each comprise one or several circuit components. Catheter 26 is connected to console 46 via an electrical interface 48, such as a port or socket. RF signals are carried from signal generator 44 to distal assembly 30, and electrophysiological signals are carried from the distal assembly to sensing circuitry 43, both via interface 48 and electrical wires (not shown) running through catheter 26.

Processor 42 receives from physician 22 (or another operator), prior to and/or during the ablation procedure, setup parameters for the procedure. For example, using one or more suitable input devices, such as a keyboard, mouse, or touch screen (not shown), physician 22 defines the electrical and temporal parameters of the RFA signals to be applied to some or all of the segments of electrodes 34. Processor 42 passes suitable control signals to signal generator 44 for performing the RFA. Processor 42 also instructs sensing circuitry 43 to acquire electrophysiological signals from certain segments of electrodes 34, as will be further detailed in FIG. 2.

Processor 42 may be further configured to track the respective positions of electrodes 34 during the RFA procedure and during electrophysiological signal acquisition, using any suitable tracking technique. For example, distal assembly 30 may comprise one or more electromagnetic position sensors (not shown), which, in the presence of an external magnetic field generated by one or more magnetic-field generators 50, output signals that vary with the positions of the sensors. Based on these signals, processor 42 may ascertain the positions of electrodes 34. Magnetic-field generators 50 are connected to console 46 via cables 52 and an interface 54. Alternatively, for each electrode 34, processor 42 may ascertain the respective impedances between the electrode and multiple external electrodes 56 on the body surface of subject 24 at various different locations, and then compute the ratios between these impedances, these ratios being indicative of the electrode's location. As yet another alternative, the processor may use both electromagnetic tracking and impedance-based tracking, as described, for example, in U.S. Pat. No. 8,456,182, whose disclosure is incorporated herein by reference.

In some embodiments, processor 42 displays, on a display screen 58, a relevant image 60 of the subject's anatomy, annotated, for example, to show the current position and orientation of distal assembly 30. Alternatively or additionally, processor 42 may display on screen 58 a map of the electrophysiological signals acquired through electrodes 34.

Processor 42, sensing circuitry 43, and electric signal generator 44 may typically comprise both analog and digital elements. Thus, sensing circuitry 43 may comprise multiple inputs with respective analog-to-digital converters (ADCs) for receiving analog electrophysiological signals from catheter 26 and for converting them to digital form for passing them to processor 42. Electric signal generator 44 typically comprises RF analog circuits for generating the RF signals for ablation, as well as digital-to-analog converters (DACs) for receiving digital control signals from processor 42.

Alternatively, the electrophysiological signals and/or control signals may be passed between processor 42 and sensing circuitry 43 and electric signal generator 44, respectively, in an analog form, provided that processor 42 is configured to send and/or to receive analog signals.

Furthermore, processor 42 typically comprises digital filters for extracting signals at given frequencies from the received electrophysiological signals.

Typically, the functionality of processor 42, as described herein, is implemented at least partly in software. For example, processor 42 may comprise a programmed digital computing device comprising at least a central processing unit (CPU) and random access memory (RAM). Program code, including software programs, and/or data are loaded into the RAM for execution and processing by the CPU. The program code and/or data may be downloaded to the processor in electronic form, over a network, for example. Alternatively or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

At the start of the RFA procedure, physician 22 inserts catheter 26 through a sheath 62 via the vascular system of subject 24 into heart 27, with balloon 32 in a collapsed configuration. Only after the catheter exits the sheath is the balloon inflated to its intended functional shape with a fluid that flows into the balloon through shaft 28. This functional shape is shown in insets 36 and 38. By containing balloon 32 in a collapsed configuration, sheath 62 also serves to minimize vascular trauma while the balloon is brought to the target location. Physician 22 navigates catheter 26 to a target location in heart 27 of subject 24, by manipulating the catheter, using a manipulator 64 near the proximal end of the catheter, and/or deflection from sheath 62. Physician 22 brings distal assembly 30 into contact with tissue, such as myocardial tissue, of heart 27. Next, under the control of physician 22 and processor 42, electrical signal generator 44 generates RFA signals, which are carried through catheter 26 in parallel to the segments of electrodes 34.

In a unipolar RFA, the currents of ablation signals flow between ablation electrodes 34 and an external electrode, or “return patch” 66, which is coupled externally between subject 24, typically on the skin of the subject's torso, and generator 44. In a bipolar RF ablation the currents of the signals flow between pairs of ablation electrodes 34.

Processor 42 acquires, either simultaneously with or alternating with the RFA, electrophysiological signals received separately and independently by selected segments of electrodes 34 from tissue of subject 24. The electrophysiological signals are carried from electrodes 34 through catheter 26 to processor 42.

Notwithstanding the particular type of ablation procedure illustrated in FIG. 1, the principles of the present invention may be applied to any suitable type of multi-channel radio-frequency ablation procedure.

FIG. 2 is a schematic detail view of the distal end of catheter 26, in accordance with an embodiment of the invention.

As described above, catheter 26 comprises shaft 28 (with only a section shown here) and distal assembly 30. Distal assembly 30 comprises balloon 32 and electrodes 34 at different respective locations on the surface of the balloon. Balloon 32 has a polar axis 106 coinciding with a longitudinal axis 104 of a distal end 102 of shaft 28. A plurality of flexible circuit substrates 105 are disposed on the expandable member about longitudinal axis 104. On each substrate 105, there is provided electrode 34. As shown, there are a plurality of electrode members (designate individually as 34) for each substrate 105. Each of electrodes 34 is divided into segments 114 along longitudinal isolation lines 108 and latitudinal isolation lines 110 (wherein “longitudinal” and “latitudinal” are defined with reference to polar axis 106). For example, electrode 34a (one of electrodes 34), shown in greater detail in an inset 112, is divided into six segments 114a, 114b, 114c, 114d, 114e, and 114f. Four of the segments, 114a-114d, have the same (or nearly same) area, whereas segments 114e and 114f are smaller than segments 114a-114d, each having an area that is, for example, approximately a quarter (¼) of the area of each of segments 114a-114d. Each segment 114 (i.e., 114a, 114b, 114c or 114d) is connected individually to a respective wire or other conductor such as electrical traces (not shown), which passes through shaft 28 to console 46, thus enabling sensing circuitry 43 and electrical signal generator 44 to address the segments individually or in parallel for purposes of sensing and ablation, as explained above. That is, each of the larger electrode segments 116a, 116b, 116c, 116d and smaller electrode segments 116e and 116f are electrically insulated from each other on the expandable member.

Electrode 34a comprises irrigation apertures 116a, 116b, 116c, and 116d, each passing through a respective segment 114a, 114b, 114c, and 114d, providing paths for fluid to flow out of balloon 32 to irrigate the tissue contacted by and in the vicinity of the respective segment. However, the two smaller segments 114e and 114f typically do not have irrigation apertures and may be irrigated by apertures 116c and 116d, for example. In alternative embodiments the smaller segments may also have irrigation apertures, as well.

In other embodiments, the number of segments of each electrode 34 may be more or less than six. Additionally or alternatively, the ratio between the areas of the larger and smaller segments may be different from 4:1 (the numeral “4” indicating that the larger segment is approximately 4 times that of the smaller segment), but it is typically at least 2:1; and the number of irrigation apertures may be different from one for the larger segments. Furthermore, although FIG. 2 shows electrodes 34 divided into segments along longitudinal and latitudinal lines 108 and 110, the division may be implemented by only longitudinal lines or by only latitudinal lines. The dividing lines may also have a different geometry, such as, for example a non-90 degree angle with respect to latitudinal lines 110.

For the purpose of ablation using electrode 34a, processor 42 commands signal generator 44 to apply an RF signal with an amplitude sufficient to ablate the tissue contacted by the electrode. The RF signal is applied simultaneously in parallel to all or some of segments 114a-114f so as to provide a sufficiently large conducting area for RF current to be passed through without damage to electrode 34a.

For the purpose of acquiring an electrophysiological signal using electrode 34a, processor 42 connects sensing circuitry 43 individually to one or more of segments 114a-114f, for example to the smaller segments 114e-114f. Thus, the conducting area through which the electrophysiological signal is acquired is sufficiently small to prevent the signals to be averaged over a wide area of the tissue. The electrophysiological signals may be acquired in this fashion concurrently from multiple segments, as well as multiple different electrodes.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A medical apparatus, comprising:
a probe comprising:
an insertion tube configured for insertion into a body cavity of a patient;
an expandable member extending along a longitudinal axis, which is connected distally to the insertion tube and is configured to be expanded about the longitudinal axis inside an organ;
a plurality of flexible circuit substrates disposed radially about the longitudinal axis at different respective locations on the expandable member; and
a plurality of ablation electrodes, each ablation electrode being disposed on a respective flexible circuit substrate of the plurality of flexible circuit substrates such that each ablation electrode is disposed on a different flexible circuit substrate and are configured to contact tissue within the body cavity, each ablation electrode being divided into (i) a first segment comprising a first area and (ii) a second segment comprising a second area by a first isolation line such that the first segment and second segment are disposed a distance from each other in a circumferential direction of the expandable member that is transverse to the longitudinal axis; and
an electrical signal generator, which is configured to apply ablation signals simultaneously in parallel to the first segment and the second segment of each ablation electrode with an amplitude sufficient to ablate the tissue contacted by one or more of the plurality of electrodes, the first segment and the second segment each being connected to the electric signal generator such that the first segment and the second segment are each individually capable of applying the ablation signals to ablate the tissue contacted by the ablation electrode.

2. The medical apparatus of claim 1, the first segment and the second segment of each ablation electrode being entirely disposed on the respective flexible circuit, an outer longitudinal edge of the first segment and an outer longitudinal edge of the second segment of each ablation electrode being inset relative to outer edges of the respective flexible circuit.

3. The medical apparatus of claim 2,
a first latitudinal edge of the first segment and a first latitudinal edge of the second segment of each ablation electrode being defined by a second isolation line,
a second latitudinal edge of the first segment and a second latitudinal edge of the second segment of each ablation electrode being defined by a third isolation line,
an inner longitudinal edge of the first segment and an inner longitudinal edge of the second segment of each ablation electrode being defined by the first isolation line,
the first segment of each ablation electrode comprising a first perimeter defined by the outer longitudinal edge of the first segment, the first latitudinal edge of the first segment, the inner longitudinal edge of the first segment, and the second latitudinal edge of the first segment, and
the second segment of each ablation electrode comprising a second perimeter defined by the outer longitudinal edge of the first segment, the first latitudinal edge of the first segment, the inner longitudinal edge of the first segment, and the second latitudinal edge of the first segment.

4. The medical apparatus of claim 1, each ablation electrode being divided into at least four segments comprising the first segment, the second segment, a third segment having a third area, and a fourth segment having a fourth area, such that the first area and the second area are each at least twice the third area and at least twice the fourth area.

5. The medical apparatus of claim 4, each ablation electrode further comprising a plurality of irrigation apertures passing through some, but not all, of the at least four segments.

6. The medical apparatus of claim 4, each ablation electrode being divided into at least six segments comprising the first segment, the second segment, the third segment, the fourth segment, a fifth segment, and a sixth segment, such that (i) the first segment is surrounded on four sides by the second segment, the third segment, the fifth segment and the respective flexible circuit and (ii) the second segment is surrounded on four sides by the first segment, the fourth segment, the sixth segment and the respective flexible circuit.

7. The medical apparatus of claim 1, further comprising sensing circuitry, which is configured to acquire electrophysiological signals from at least one of the first segment or the second segment of each ablation electrode separately and independently of the other of the first segment or the second segment of each ablation electrode.

8. The medical apparatus of claim 7, each ablation electrode being divided into at least four segments comprising the first segment, the second segment, a third segment having a third area, and a fourth segment having a fourth area, such that the first area and the second area are each larger than the third area and larger than the fourth area, and the medical apparatus further comprising a processor configured to connect the sensing circuitry individually to the third segment and to the fourth segment of each ablation electrode.

9. The medical apparatus of claim 7, further comprising:
a console that comprises the electrical signal generator and the sensing circuitry,
each ablation electrode having a first conductor connected to the first segment and a second conductor connected to the second segment, the first conductor and second conductor passing through the insertion tube to the console such that (i) the electrical signal generator is capable of applying ablation signals simultaneously in parallel to the first segment and the second segment via the first conductor and the second conductor and (ii) the sensing circuitry is capable of acquiring electrophysiological signals from at least one of the first segment or the second segment of each ablation electrode separately and independently of the other of the first segment or the second segment of each ablation electrode via the first conductor or the second conductor.

10. The medical apparatus of claim 1, the electrical signal generator being configured to apply bipolar ablation signals simultaneously in parallel to the first segment and the second segment of a first ablation electrode of the plurality of ablation electrodes, the bipolar ablation signals being configured to flow to the first segment and the second segment of a second ablation electrode of the plurality of ablation electrodes.

11. A method for medical treatment and diagnostics, the method comprising:
providing a probe for insertion into a body cavity of a patient, the probe comprising:
an insertion tube configured for insertion into a body cavity of a patient;
an expandable member extending along a longitudinal axis, which is connected distally to the insertion tube and is configured to be expanded about the longitudinal axis inside an organ;
a plurality of flexible circuit substrates disposed radially about the longitudinal axis at different respective locations on the expandable member; and
a plurality of ablation electrodes, each ablation electrode being disposed on a respective flexible circuit substrate of the plurality of flexible circuit substrates such that each ablation electrode is disposed on a different flexible circuit substrate and are configured to contact tissue within the body cavity, each ablation electrode being divided into (i) a first segment comprising a first area and (ii) a second segment comprising a second area by a first isolation line such that the first segment and second segment are disposed a distance from each other in a circumferential direction of the expandable member that is transverse to the longitudinal axis;
expanding the expandable member within the body cavity so that one or more of the ablation electrodes contact tissue within the body cavity; and
applying ablation signals simultaneously in parallel to the first segment and the second segment of at least one ablation electrode of the plurality of ablation electrodes with an amplitude sufficient to ablate the tissue contacted by the one or more of the ablation electrodes, each of the first segment and the second segment being connected to an electric signal generator such that each of the first segment and the second segment is individually capable of applying the ablation signals to ablate the tissue contacted by the one or more of the ablation electrodes.

12. The method of claim 11, comprising:
entirely disposing on the respective flexible circuit the first segment and the second segment of each ablation electrode, and
insetting an outer longitudinal edge of the first segment and an outer longitudinal edge of the second segment of each ablation electrode relative to outer edges of the respective flexible circuit.

13. The method of claim 12, further comprising:
defining a first latitudinal edge of the first segment and a first latitudinal edge of the second segment of each ablation electrode by a second isolation line,
defining a second latitudinal edge of the first segment and a second latitudinal edge of the second segment of each ablation electrode by a third isolation line,
defining an inner longitudinal edge of the first segment and an inner longitudinal edge of the second segment of each ablation electrode by the first isolation line,
the first segment of each ablation electrode comprising a first perimeter defined by the outer longitudinal edge of the first segment, the first latitudinal edge of the first segment, the inner longitudinal edge of the first segment, and the second latitudinal edge of the first segment, and
the second segment of each ablation electrode comprising a second perimeter defined by the outer longitudinal edge of the first segment, the first latitudinal edge of the first segment, the inner longitudinal edge of the first segment, and the second latitudinal edge of the first segment.

14. The method of claim 11, further comprising dividing each ablation electrode into at least four segments comprising the first segment, the second segment, a third segment having a third area, and a fourth segment having a fourth area, such that the first area and the second area are each at least twice the third area and at least twice the fourth area.

15. The method of claim 14, further comprising passing a plurality of irrigation apertures through some, but not all, of the at least four segments of each ablation electrode.

16. The method of claim 14, further comprising dividing each ablation electrode into at least six segments comprising the first segment, the second segment, the third segment, the fourth segment, a fifth segment, and a sixth segment, such that (i) the first segment is surrounded on four sides by the second segment, the third segment, the fifth segment and the respective flexible circuit and (ii) the second segment is surrounded on four sides by the first segment, the fourth segment, the sixth segment and the respective flexible circuit.

17. The method of claim 11, further comprising:
acquiring electrophysiological signals, via sensing circuitry, from at least one of the first segment or the second segment of the at least one ablation electrode of the plurality of ablation electrodes separately and independently of the other of the first segment or the second segment of each ablation electrode.

18. The method of claim 17, further comprising:
dividing each ablation electrode into at least four segments comprising the first segment, the second segment, a third segment having a third area, and a fourth segment having a fourth area, such that the first area and the second area are each larger than the third area and larger than the fourth area, and
connecting, via a processor, the sensing circuitry individually to the third segment and to the fourth segment of each ablation electrode.

19. The method of claim 11, further comprising:
applying bipolar ablation signals simultaneously in parallel to the first segment and the second segment of a first ablation electrode of the plurality of ablation electrodes such that the bipolar ablation signals flow to the first segment and the second segment of a second ablation electrode of the plurality of ablation electrodes.

20. The method of claim 11, further comprising:
connecting a first conductor to the first segment of one of the ablation electrodes of the plurality of ablation electrodes;
connecting a second conductor to the second segment of the one of the ablation electrodes of the plurality of ablation electrodes;
routing the first conductor and second conductor through the insertion tube to a console that comprises the electrical signal generator and the sensing circuitry such that (i) the electrical signal generator is capable of applying ablation signals simultaneously in parallel to the first segment and the second segment via the first conductor and the second conductor and (ii) the sensing circuitry is capable of acquiring electrophysiological signals from at least one of the first segment or the second segment of each ablation electrode separately and independently of the other of the first segment or the second segment of each ablation electrode via the first conductor or the second conductor.
